(19) United States
(12) Patent Application Publication
(10) Pub. No.: US 2023/0097980 A1
(43) Pub. Date: Mar. 30, 2023

(54) CEREBRAL DURAL VENOUS SINUS STENT

(71) Applicant: The Regents of the University of California, Oakland, CA (US)

(72) Inventor: Matthew Amans, San Francisco, CA (US)

(73) Assignee: The Regents of the University of California, Oakland, CA (US)

(21) Appl. No.: 17/908,623

(22) PCT Filed: Mar. 2, 2021
(86) PCT No.: PCT/US2021/020458

§ 371 Date: Sep. 1, 2022

Related U.S. Application Data
(60) Provisional application No. 62/984,549, filed on Mar. 3, 2020.

(51) Int. Cl. A61F 2/852; A61F 2/95

(52) U.S. Cl. CPC A61F 2/852; A61F 2/95

(57) ABSTRACT

An implantable device includes a tubular member defining a longitudinal axis and a lumen. The tubular member includes plurality of filaments defining a plurality of openings therebetween; a distal end portion having a distal diameter; a proximal end portion having a proximal diameter that is larger than the distal diameter; and an intermediate portion having an intermediate diameter that is smaller than the distal diameter.

### CROSS-REFERENCE TO RELATED APPLICATION

The present application claims priority to and the benefit of U.S. Patent Provisional Application No. 62/984,549, filed on Mar. 3, 2020. The entire disclosure of the foregoing application is incorporated by reference herein.

### BACKGROUND

Idiopathic intracranial hypertension (IIH) is a common disorder afflicting young overweight women in which elevated intracranial pressure can lead to blindness and cognitive decline as well as severe symptoms of headache and pulsatile tinnitus (PT). Dural venous sinus stenting is an emerging therapy for IIH patients and PT patients with venous sinus stenoses. In order to be eligible for therapy, venous manometry is performed and a sufficient pressure gradient across the stenosis has to be measured (typically greater than 5 or 8 mmHg) with patients under little to no sedation. However, the installation of the currently available stents, which are designed either for carotid arteries or peripheral venous applications is quite cumbersome for operators and painful for patients due to the stiffness and high radial force of carotid stents. Thus, the stent procedure typically requires general anesthesia to perform safely. High radial forces imparted by carotid stents may also cause severe headaches in patients that can sometimes only be treated with steroids. Steroids in IIH patients are quite dangerous as cessation of the steroids can itself exacerbate the underlying IIH condition.

Currently available stents, e.g., typical carotid stents, also do not come in lengths or configurations suitable for treating IIH. In particular, carotid stents do not come in sufficiently long constructs, do not have suitable diameters, and are primarily round in shape. In response, many operators are using multiple stent constructs that are widely discrepant in size. This exposes patients to increased procedural risk, potentially mismatching or undersizing the stents, which can lead to stent migration (in cases where undersized stents are used) or headaches (in cases where oversized stents are used).

In addition, many patients with PT have a venous sinus stenosis, which is the source of their PT. However, many sinus stenoses are asymptomatic. The only currently available stents for treating transverse sinus stenoses are permanent implants that need to be placed with patients under general anesthesia. A stent that can be safely and painlessly positioned while patients are awake would allow them to report if their symptoms are improved immediately upon deployment of the stent. In patients whose symptoms are exacerbated by stenting, or not improved, the current technology does not allow for removal of the stent.

In addition, many patients treated with currently available stents have to undergo revision surgery due to failure of the initial stenting procedure to durably treat venous hypertension and IIH. This may be in part because the utilized carotid stents have too high of a radial force and are circular. These stents decompress the dural venous sinuses to such a point where normal fluctuations in the intracranial pressure can cause the dural venous sinus that have not been treated with a stent to collapse. In other words, the venous sinuses are unable to resist the normal transient spikes in intracranial pressure. The venous sinuses' ability to resist compression from the intracranial pressure is a combination of the pressure within the veins and the intrinsic resistive force of the sinuses. By placing a stent with very high resistive forces within the dural venous sinuses, the venous system does not have the ability to withstand the normal transient spikes in intracranial pressure, and thus IIH recurs in a large number of patients treated with venous sinus stenting.

Accordingly, there is a need for improved treatment methods and devices that address the shortcomings of conventional stents.

### SUMMARY

The present disclosure provides a stent configured and designed for the unique environment of the dural venous sinuses and in particular sigmoid sinus and torcula segments of the cerebral dural vein. The stent may include a flexible proximal tip that may be tapered to easily and nearly painlessly traverse the venous sinuses and the stenosis. The disclosed stent has a low radial force sufficient to open a venous sinus stenosis, which may be from about 0.1 Newton per square millimeter (N/mm²) to about 0.2 N/mm².

As used herein the term “distal” refers to the portion of an implantable device that is further from the heart, while the term “proximal” refers to the portion that is closer to the heart. Thus, with respect to the blood flow through a vein, blood flows from a distal end to a proximal end. Accordingly, the proximal portion may be disposed adjacent a sigmoid sinus and the distal portion may be disposed adjacent a torcula after implantation.

As used herein, the terms “biodegradable” and “bioabsorbable” are used with respect to a property of a material. “Biodegradable” is a material that is capable of being decomposed or broken down in vivo and subsequently excreted. “Bioabsorbable” is a material that is capable of being decomposed or broken down in vivo and subsequently resorbed. Both biodegradable and bioabsorbable materials are suitable for purposes of this application and thus for simplicity, unless otherwise directed, biodegradable materials and bioabsorbable materials are collectively referred to as “biodegradable” herein. Conversely, “non-biodegradable” is a biocompatible (i.e., not harmful to living tissue) material is not decomposed or broken down in vivo. In addition, the term “dissolution” as used in the description refers to the breakdown of both biodegradable and bioabsorbable materials.

The stent's radial force is such that it is greater when the stent is collapsed, and lesser when the stent is expanded. Such a design allows for the stent to temporarily narrow due to normal transient increases in the intracranial pressure up to a point, but then resist further compression. By compressing in response to the increased intracranial pressures (ICP), the stent would cause temporary venous hypertension to resist collapse of the untreated dural venous sinuses during the transient changes in ICP. When the transient ICP increase resolves, i.e., ICP decreases, the stent would re-expand. Conventional stents do not change their expansion size in response to changes in the ICP due to high radial force (or high crush resistive force). As a result, a conventional stent may expand a vessel beyond its natural diameter resulting in a wider portion. At the nexus of the unstented and stented portion of the blood vessel, blood flow may result in turbulent blood flow and a resultant pressure drop. Thus, conventional stents may fail at that juncture.

The stent according to the present disclosure may have any suitable cross-section, e.g., oval, circular, triangular, rectangular, polygonal, etc. to suit the geometry of the vessel, i.e., dural venous sinuses. The stent may have a length from about 30 mm to about 200 mm and may taper from a proximal portion (i.e., larger diameter) to a distal portion (i.e., smaller diameter). A proximal diameter may be from about 8 mm to about 14 mm and a distal diameter may be from about 4 mm to about 8 mm. After implantation, the proximal portion may be disposed adjacent to or within the sigmoid sinus. The distal portion may be disposed adjacent the torcula or within the superior sagittal sinus. The tapered portion minimizes the change in the blood vessel shape and cross-sectional area, limiting generation of turbulent flow.

A secondary stent may also be used to treat the unique anatomy considerations of the posterior third of the superior sagittal sinus. The diameter may be from about 4 mm to about 5 mm throughout its length and may have the ability to flare wider to accommodate the torcula. It may also be tapered from about 3 mm distally to about 6 mm proximally. It may taper to be similar cross-sectional area to the native sinus. It may also then have a flare to a wider diameter to accommodate the torcula. The secondary stent may be from about 60 to 100 mm in length.

The stent may have a closed cell or braided design allowing for the stent to be retrievable since such a structure allows for reversible expansion and collapse of the stent. In embodiments, the stent may have an open cell design to minimize radial force. In embodiments, the stent may be mounted to a wire to facilitate retrievability. In further embodiments, the stent may have a hook construct on the side of the stent close to the jugular vein to allow operators to retrieve the stent. Pulling the hook adjusts dimensions and shape of the stent, i.e., change the shape of the taper. The hook also allows for the stent to be recaptured by a catheter having a counterpart hook. The stent may be formed from a biodegradable material such that the stent dissolves after a certain period of time, i.e., once the stent has “healed” into position. The stent may be formed from a degradable material such that after a period of time if the stent is no longer required, a reagent, or chemical, or other material can be injected within the stent, adjacent to the stent, or systemically that causes the stent to dissolve, or degrade.

The disclosed stent may be used in safer, less painful, and more durable treatment for IIH and PT. IIH affects 20 in 100,000 overweight women of childbearing age. As the obesity epidemic increases, this patient population is expected to continue to increase. Most of these patients can be well treated with a venous sinus stent according to the present disclosure. The alternative conventional therapies have significant limitations including poor safety records, high revision therapy rates, or difficulties with patient tolerances. PT afflicts between 3 and 5 million Americans, and has very high comorbid associations with depression, anxiety and even suicidal ideations. There are very few conventional effective treatments for PT.

According to one embodiment of the present disclosure, an implantable device is disclosed. The implantable device includes a tubular member defining a longitudinal axis and a lumen. The tubular member includes a plurality of filaments defining a plurality of openings therebetween; a distal end portion having a distal diameter; a proximal end portion having a proximal diameter that is larger than the distal diameter; and an intermediate portion having an intermediate diameter that is smaller than the distal diameter.

According to one aspect of the above embodiment, the proximal diameter is from about 10 mm to about 14 mm. The distal diameter is from about 4 mm to about 8 mm. The intermediate diameter is from about 4 mm to about 7 mm. The proximal diameter may be larger than the distal diameter by a factor from about 2 to about 3.

According to another aspect of the above embodiment, the implantable device further includes an attachment member including a plurality of attachment filaments and a hook coupled to the attachment filaments. Rotation of the attachment member about the longitudinal axis in a first direction expands the tubular member and rotation in a second direction, opposite the first direction, constrains the tubular member. The tubular member is formed from a non-biodegradable material and the attachment member is formed from a biodegradable material.

According to another aspect of the above embodiment, the implantable device further includes a wire disposed within and through the lumen and may be parallel to the longitudinal axis, wherein the wire is coupled to the tubular member. The tubular member is formed from a non-biodegradable material and the wire is formed from a biodegradable material. According to a further aspect of the above embodiment, the tubular member is formed from a biodegradable material.

According to a further embodiment of the present disclosure, a method for treating a cerebral dural venous sinus is disclosed. The method includes inserting an implantable device into the cerebral dural venous sinus. The implantable device includes a tubular member defining a longitudinal axis and a lumen. The tubular member includes a plurality of filaments defining a plurality of openings therebetween; a distal end portion having a distal diameter; a proximal end portion having a proximal diameter that is larger than the distal diameter; and an intermediate portion having an intermediate diameter that is smaller than the distal diameter.

According to one aspect of the above embodiment, the proximal end portion is disposed adjacent a sigmoid sinus of the cerebral dural venous sinus. The distal end portion is disposed adjacent a torcula of the cerebral dural venous sinus. The stent may also be long enough such that the distal end of the portion is disposed in the superior sagittal sinus.

According to another aspect of the above embodiment, the implantable device further includes an attachment member including a plurality of attachment filaments and a hook coupled to the attachment filaments. The method also includes rotating the attachment member about the longitudinal axis in a first direction to expand the tubular member. The method further includes rotating the attachment member about the longitudinal axis in a second direction, opposite the first direction to constrain the tubular member.

According to a further aspect of the above embodiment, the tubular member is formed from a non-biodegradable material and the attachment member is formed from a biodegradable material. The method further includes injecting a reagent into the cerebral dural venous sinus to dissolve at least a portion of the attachment member.

According to yet another aspect of the above embodiment, the proximal diameter is from about 10 mm to about 14 mm, the distal diameter is from about 4 mm to about 8 mm, and the intermediate diameter is from about 4 mm to about 7 mm.

According to a further embodiment of the present disclosure, an implantable device is disclosed. The implantable device includes a plurality of tubular members disposed in a parallel relative to each other and defining a longitudinal axis and a lumen. Each of the tubular members has a crush resistive force equal to an intracranial pressure threshold, such that each of the tubular members is configured to collapse in response to intracranial pressure increasing above the threshold and expanding in response to the intracranial pressure dropping below the threshold. In other words, each of the tubular members has a different threshold pressure above which it collapses. One collapses with high normal physiological ranges of ICP. One collapses with fairly high ICP, and one is essentially always patent.

According to yet another embodiment of the present disclosure, an implantable device is disclosed. The implantable device includes a first expandable tubular member having a crush resistive force equal to a first intracranial pressure threshold, such that the first expandable tubular member is configured to collapse in response to intracranial pressure increasing above the first intracranial pressure threshold and expanding in response to the intracranial pressure dropping below the first intracranial pressure threshold. The implantable device further includes a second expandable tubular member contacting the second expandable tubular member and disposed in parallel thereto, the second expandable tubular member having a crush resistive force equal to a second intracranial pressure threshold, such that the second expandable tubular member is configured to collapse in response to intracranial pressure increasing above the second intracranial pressure threshold and expanding in response to the intracranial pressure dropping below the second intracranial pressure threshold.

According to one aspect of the above embodiment, the first intracranial pressure threshold and the second intracranial pressure threshold are different.

### BRIEF DESCRIPTION OF THE DRAWINGS

Embodiments of the present disclosure are described herein with reference to the accompanying drawings, wherein:

FIG. 1 is a perspective view of an implantable device according to one embodiment of the present disclosure;

FIG. 2 is a perspective view of an implantable device according to another embodiment of the present disclosure;

FIG. 3 is a perspective view of an implantable device according to a further embodiment of the present disclosure; and

FIG. 4 is a perspective view of an attachment member of the implantable device of FIG. 1 according to one embodiment of the present disclosure;

FIG. 5 is a perspective view of an implantable device according to a further embodiment of the present disclosure;

FIG. 6 is a perspective view of an implantable device according to yet another embodiment of the present disclosure; and

FIG. 7 is a perspective view of an implantable device according to one further embodiment of the present disclosure.

### DETAILED DESCRIPTION

Embodiments of the present disclosure are described in detail with reference to the drawings, in which like reference numerals designate identical or corresponding elements in each of the several views.

The present disclosure provides a method for treating IIH and PT by catheterizing the cerebral venous sinuses and implanting a device. Suitable implantable devices according to the present disclosure may be self-expanding or balloon expandable stents having an outer wall of varying diameters.

The implantable devices may be constrained in a catheter, and when un-sheathed at the target location within the target vein or any other vascular location, self-expand so as to contact and push against the vessel walls to prevent migration of the device. In embodiments, the device may include one or more attachment members, e.g., hooks, anchors, or teeth, to embed the device in the venous wall. The outer walls of the implantable device are sufficiently permeable so as not to impede venous ingress from the cortical veins or internal jugular vein into the larger sinus. Thus, the device is minimally thrombogenic in order to minimize embolic risk to the systemic venous circulation and the pulmonary arterial system as a whole, since thrombogenicity could result in parent venous sinus occlusion.

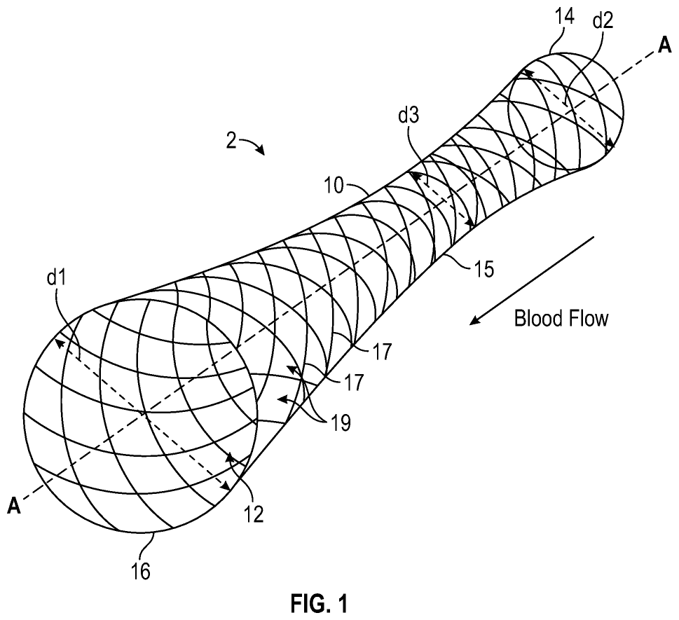

With reference to FIGS. 1-3, an implantable device 2, e.g., stent, according to the present disclosure includes a tubular member 10 defining a longitudinal axis “A-A” and a lumen 12 extending along the longitudinal axis “A-A.” The tubular member 10 includes a distal end portion 14, and a proximal end portion 16. The tubular member 10 includes a plurality of interconnected filaments 17 defining a plurality of openings 19 in between the interconnected filaments 17. The tubular member 10 is configured to contact the walls of a vessel such as a dural venous sinus.

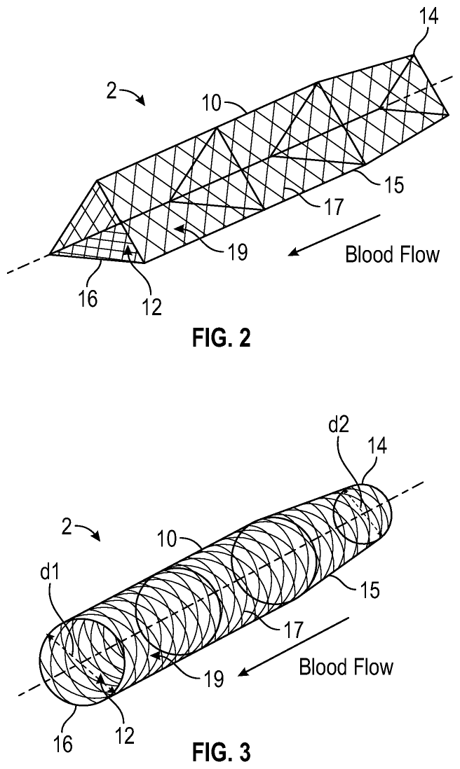

Following implantation, the distal end portion 14 may be disposed adjacent a torcula and the proximal end portion 16 adjacent a sigmoid sinus after implantation. The tubular member 10 may have any suitable cross-sectional shape to match a native shape of a blood vessel, such as oval, circular, polygonal, (i.e., triangular or rectangular). As shown in FIG. 2, the tubular member 10 may have a triangular cross-section, which more closely approximates certain vessel shapes, than a circular tubular member 10. As noted above, a mismatch in geometries between stents and blood vessels may result in generating turbulence.

In further embodiments, the proximal end portion 16 may have a proximal cross-sectional shape, whereas the distal end portion 14 may have a distal cross-sectional shape that is different from the first cross-sectional shape to allow for a better fit. The proximal cross-sectional shape may be triangular and the distal cross-sectional shape may be rectangular, oval, or circular to better fit within the sigmoid sinus.

The radial force of the tubular member 10 may also be characterized as crush resistive force force, namely, the force needed to collapse the tubular member 10, and chronic radial outward force, namely, the chronic pressure exerted by the tubular member 10 when in nominal state (i.e., expanded configuration). At nominal state, the radial force may be from about 0 mmHg and 100 mmHg, and in embodiments, the radial force may be from about 10 mmHg to about 30 mmHg. The chronic radial outward force at nominal may be from about 0 mmHg to about 30 mmHg, and in embodiments may be from about 0 mmHg to about 10 mmHg. Radial resistive force at approximately 30% of the nominal state may be from about 20 mmHg to about 70 mmHg, and in embodiments may be from about 30 mmHg to about 50 mmHg. Chronic radial outward force at approximately 30% nominal may be from about 15 mmHg to about 70 mmHg, and in embodiments may be from about 20 mmHg to about 50 mmHg. Radial force when the tubular member 10 is fully constrained may be from about 30 mmHg to about 200 mmHg, and in embodiments may be from about 40 mmHg to about 60 mmHg. The radial force when the tubular member 10 is expanded is sufficient to withstand intracranial pressure fluctuations and minimizes the risks of migration but low enough such that nominal radial force does not cause dural irritation.

The tubular member 10 may have a length from about 30 mm to about 200 mm. The tubular member 10 may have a tapered shape as shown in FIG. 3, such that a proximal diameter d1 of the proximal end portion 16 is larger than a distal diameter d2 of the distal end portion 14. The proximal diameter d1 may be from about 10 mm to about 14 mm and the distal diameter d2 may be from about 4 mm to about 8 mm. In embodiments, the proximal diameter d1 may be larger than the distal diameter d2 by a factor from about 2 to about 3.

As shown in FIG. 1, the tubular member 10 may have an hour-glass shape having an intermediate portion 15 with an intermediate diameter d3 that is smaller than the distal diameter d2 and the proximal diameter d1. The flared design of the hour-glass shape also allows the tubular member 10 to withstand intracranial pressure fluctuations and minimizes the risks of migration. The intermediate diameter d3 may be from about 4 mm to about 7 mm. With respect to FIG. 2, where the cross-sectional shape of the tubular member 10 is not circular, tapering may be achieved by decreasing width or other cross-sectional dimension to form a tapered portion, i.e., distal end portion 14.

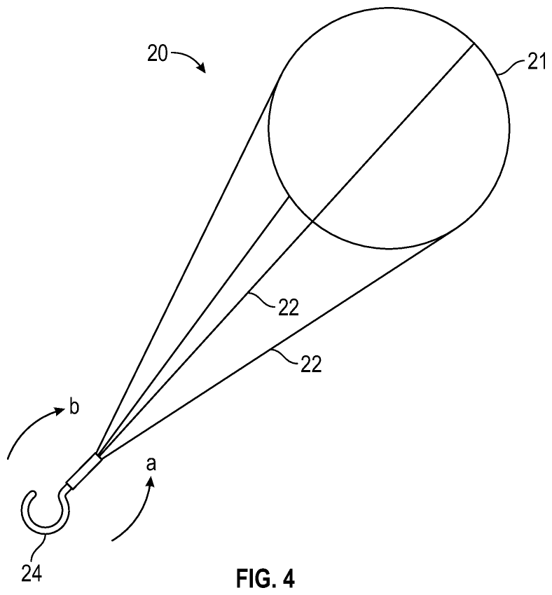

With reference to FIG. 4, the tubular member 10 may include an optional attachment member 20 coupled thereto. The attachment member 20 may include an optional loop 21 coupled to one or more attachment filaments 22. The loop 21 and/or the attachment filaments 22 may be continuous with the filaments 17 and may be woven, braided, or otherwise coupled to the tubular member 10 (FIG. 4). In embodiments, the attachment filaments 22 may be coupled to a hook 24. The loop 21 may be coupled at an intermediate location of the tubular member 10 such that the loop 21 is adjacent to the intermediate diameter d3. Pulling and/or rotating the attachment filaments 22 with the hook 24 modifies the shape of the tubular member 10 by adjusting the size of the intermediate diameter d3. In embodiments rotating the attachment filaments 22 via the hook 24 about the longitudinal axis “A-A” in either direction. Thus, rotating in a first, e.g., clockwise, direction a expands the tubular member 10 and increases the intermediate diameter d3 and rotating in a second, e.g., counterclockwise, direction b constrains the tubular member 10 and decreases the intermediate diameter d3. This would allow for more patient-specific sizing of the tubular member 10, radial force tuning, and potential removal. In further embodiments, the hook 24 and allows for an external device, such as a recapture catheter (not shown) to attach to the tubular member 10 remove the tubular member 10.

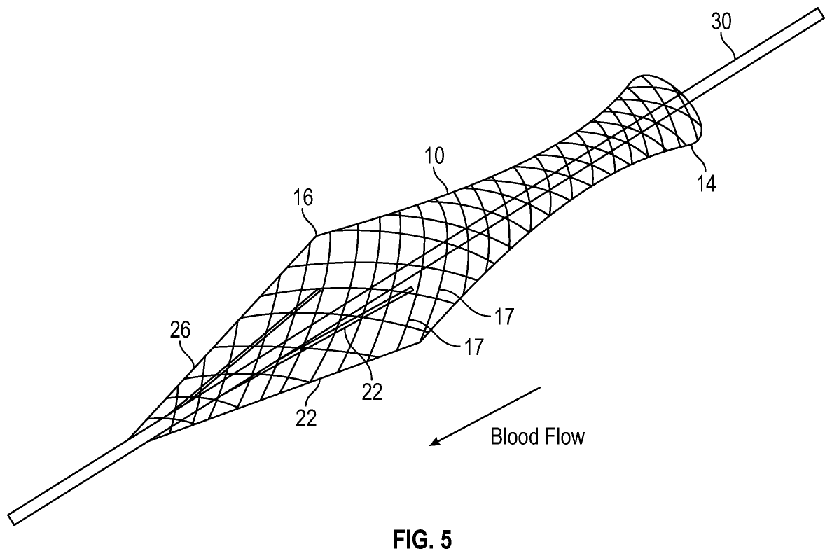

With reference to FIG. 5, the tubular member 10 may be connected to a wire 30 via the attachment filaments 22. The wire 30 is disposed within and through the lumen 12 and may be parallel to the longitudinal axis “A-A.” The wire 30 may be used in a similar manner as the hook 24 to expand or constrain the tubular member 10 by rotation such that after implantation the intermediate diameter d3 of the tubular member 10 may be adjusted. The tubular member 10 may also include a tapered proximal cone 26 coupled to the proximal end portion 16 disposed over the attachment filaments 22. The shape of the tapered proximal cone 26 provides for easy and nearly painless traversal of the venous sinuses and the stenosis.

Since various blood vessels have different blood flow parameters and properties, it would be useful to tailor the intermediate diameter d3 of the tubular member 10 according to the properties of the blood flow using the attachment filaments 22, the hook 24, and/or the wire 30. The tubular members 10 of FIGS. 1-5 may also include a plurality of attachment members, such as hooks, anchors, teeth, or other structures configured to grasp the walls of the blood vessel, such that the tubular member 10 are secured within vessel and to minimize migration of the tubular member 10 after implantation.

In embodiments, the attachment filaments 22, the hook 24, and/or the wire 30 may be removably coupled to the tubular member 10 by using a release mechanism, which may be mechanical, electrolytic, or chemical. In embodiments, the tubular member 10 may be formed from a non-biodegradable material and the attachment filaments 22, the hook 24, and/or the wire 30. Regarding a chemical release mechanism, a reagent may be injected either systemically intravenously or locally via a catheter positioned in the venous system “upstream” from the tubular member 10 to dissolve attachment points coupling the attachment filaments 22, the hook 24, and/or the wire 30 to the tubular member 10. In further embodiments, the attachment filaments 22, the hook 24, the wire 30, as well as the tubular member 10 may be formed from biodegradable material dissolution of which may be accelerated by the injected reagent to dissolve some or all of the attachment filaments 22, the hook 24, the wire 30, and/or the tubular member 10. Complete or partial dissolution would obviate the need for anti-platelet therapy and reduce radial force.

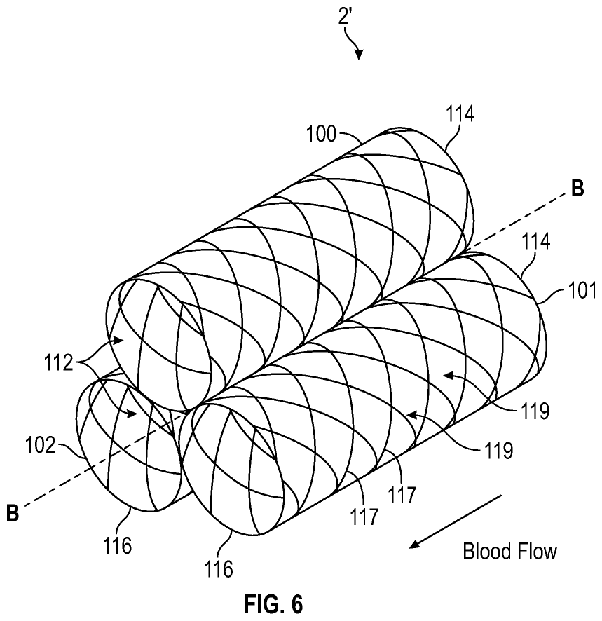

With reference to FIG. 6, another embodiment of an implantable device 2′, which includes a plurality of tubular members 100, 101, 102 arranged in a parallel configuration relative to each other, such that each of the respective longitudinal axes are parallel to each other and to a longitudinal axis “B-B”. Each of the tubular members 100, 101, 102 is substantially similar to the tubular member 10 and the differences between them are described below.

Each of the tubular members 100, 101, 102 defines a lumen 112 extending along the longitudinal axis “B-B.” The tubular members 100, 101, 102 include a distal end portion 114, and a proximal end portion 116. The tubular members 100, 101, 102 include a plurality of interconnected filaments 117 defining a plurality of openings 119 in between the interconnected filaments 117.

The tubular members 100, 101, 102 may have any suitable cross-section and dimensions as described above with respect to the tubular member 10. Each of the tubular members 100, 101, 102 may have a different crush resistive (“CR”) force. Thus, the first tubular member 100 may have a low CR force, the second tubular member 101 may have a medium CR force, and a third tubular member 102 may have a high CR force. In embodiments, the low CR force may be from about 0.002 N/mm² to about 0.004 N/mm². The medium CR force may be from about 0.003 N/mm² to about 0.006 N/mm². The high CR force may be about 0.0065 N/mm² or above.

As noted above, as ICP fluctuates, the cerebral dural vein is compressed or expanded in response to the pressure. ICP may be from about 5 mmHg to about 50 mmHg. Thus, the low CR force may be selected to correspond to a first ICP threshold, which may be from about 20 mmHg to about 30 mmHg. As the ICP begins to increase, the first tubular member 100 (i.e., low CR tubular member) is compressed and/or collapsed first, thereby resulting in a smaller diameter of the vessel since only the second tubular member 101 and the third tubular member 102 remain open. As the ICP continues to increase, the second tubular member 101 (i.e., middle CR tubular member) is also compressed and/or collapsed, resulting in further compression of the blood vessel. The middle CR force may be selected to correspond to a second ICP threshold, which may be from about 35 mmHg to about 45 mmHg. The third tubular member 102 may have a high CR, e.g., 50 mmHg or above, such that the tubular member 102 does not collapse as ICP increases. Thus, the third lumen 112 remains open.

In embodiments, the implantable device 2′ may include only two tubular members 100 and 101 or any other suitable number of tubular members, e.g., four or more. In this embodiment one of the tubular members of the implantable device 2′ has a high CR force and is configured to remain in an expanded configuration after deployment regardless of the ICP. The remaining tubular members, i.e., one or more, are configured to collapse at predetermined ICP thresholds.

The first and second tubular members 100 and 101 may be machined or laser cut from a solid tube of material to form the interconnected filaments according to the present disclosure to provide for high opening force, but relatively low CR force. The third tubular member 102 may be formed by braiding metal wire, polymer filaments, or combinations thereof, to form a tubular member having a high CR force that is impervious to high ICP.

As blood pressure increases, which occurs in response to increase in ICP, the blood vessel may recover its shape, allowing for each of the tubular members 100, 101, 102 to reform into its fully expanded configurations. In embodiments, the tubular member 10 of the implantable device 2 may have a CR force configured to collapse the tubular member 10 into its collapsible configuration once ICP reaches a predetermined threshold. Once ICP drops below the threshold, the tubular member 10 returns to its expanded configuration.

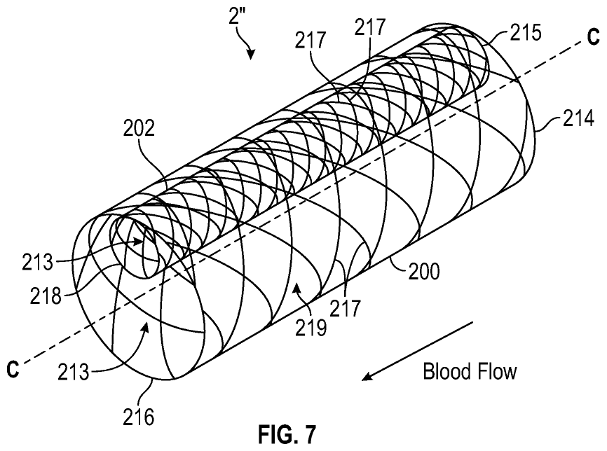

With reference to FIG. 7, yet another embodiment of an implantable device 2″ includes a plurality of tubular members 200 and 202, namely, the outer tubular member 200 and the inner tubular member 202, arranged in a parallel, nested configuration relative to each other, such that each of the respective longitudinal axes are parallel to each other and to a longitudinal axis “C-C”. Each of the tubular members 200 and 202 is substantially similar to the tubular member 10 and the differences between them are described below.

The outer tubular member 200 defines a lumen 212 extending along the longitudinal axis “C-C.” The inner tubular members 200 includes a distal end portion 214 and a proximal end portion 216. The tubular member 202 also defines a lumen 213 having a distal end portion 215 and a proximal end portion 218.

The inner tubular member 202 is coupled at one or more locations of an inner surface (i.e., filaments 217) of the outer tubular member 200, such that the inner tubular member 200 is disposed within the lumen 212. The outer tubular member 200 and inner tubular member 202 include a plurality of interconnected filaments 217 defining a plurality of openings 219 in between the interconnected filaments 217.

Each of the tubular members 200 and 202 has a different CR force. Thus, the outer tubular member 200 has a low CR force while the second tubular member 202 has a high CR force. In embodiments, the low CR force may be from about 0.002 N/mm² to about 0.004 N/mm². The high CR force may be about 0.0065 N/mm² or above.

As noted above, as ICP fluctuates the cerebral dural vein expands or contracts. Thus, the low CR force may be selected to correspond to a first ICP threshold, which may be from about 20 mmHg to about 30 mmHg. As the ICP begins to increase, the outer tubular member 200 is compressed and/or collapsed first, thereby resulting in a smaller diameter of the vessel. As the ICP continues to increase, the inner tubular member 202 has a high CR such that the tubular member 202 does not collapse as ICP continues to increase. Thus, the lumen 213 remains open.

The outer tubular member 200 may be machined or laser cut from a solid tube of material to form the interconnected filaments according to the present disclosure to provide for high opening force, but relatively low CR force. The inner tubular member 202 may be formed by braiding metal wire, polymer filaments, or combinations thereof, to form a tubular member having a high CR force that is impervious to high ICP.

The implantable devices 2, 2′, 2″ of FIGS. 1-7 may be delivered to the target vessels, e.g., cerebral or cervical veins, and in particular, to a location of maximal sound generation using any suitable transvenous surgical methods, which may include transfemoral, trans-torcular, or internal jugular vein access. Suitable delivery devices include balloon catheters and constrained stent delivery catheters depending on the type of implantable device being used.

The implantable devices 2, 2′, 2″ may be implanted within the target vessel by attaching the implantable devices 2, 2′, 2″ to the walls of the target vessels in order to align the longitudinal axes of the implantable devices 2, 2′, 2″ with the blood flow. In alternative embodiments, the implantable devices 2, 2′, 2″ may be implanted by attaching the distal end portion 14 and proximal end portion 16 to the walls of the target vessels in order to place the implantable devices 2, 2′, 2″ across the target vessels and transverse with the blood flow.

The implantable devices 2, 2′, 2″ may be self-expanding stents formed from a non-biodegradable material, such as a metal or a shape memory material, e.g., a nickel-titanium alloy (nitinol) or shape memory polymers, such as those disclosed in U.S. Pat. No. 5,954,744, the entire disclosure of which is incorporated by reference herein. The implantable devices 2, 2′, 2″ may be machined or laser cut from a solid tube of material to form the interconnected filaments according to the present disclosure. In other embodiments, the implantable devices 2, 2′, 2″ may be formed by braiding metal wire, polymer filaments, or combinations thereof, into desired shapes described above with respect to FIGS. 1-7.

In further embodiments, the implantable devices 2, 2′, 2″ may be formed from a bioabsorbable/biodegradable material that dissolves or breaks down within a vessel. Suitable biodegradable materials include synthetic and naturally derived polymers and co-polymers, as well as blends, composites, and combinations thereof. Examples of suitable materials include but are not limited to polylactide (PLA) [poly-L-lactide (PLLA), poly-DL-lactide (PDLLA)], polyglycolide (PLG or PLGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly(alpha-hydroxy acid) or two or more polymerizable monomers such as trimethylene carbonate, ε-caprolactone, polyethylene glycol, 4-tert-butyl caprolactone, N-acetyl caprolactone, poly(ethylene glycol)bis(carboxymethyl) ether, polylactic acid, polyglycolic acid, or polycaprolactone, fibrin, chitosan, or polysaccharides.

In embodiments, the implantable devices 2, 2′, 2″ may be self-expanding due to the inherent resiliency of particular biodegradable materials such as, for example, poly-L-lactide, poly-D-lactide, polyglycolide, such that filaments return to an expanded state when released from a compressed state. Each type of biodegradable polymer has a characteristic degradation rate in the body. Some materials are relatively fast-biodegrading materials (weeks to months) while others are relatively slow-biodegrading materials (months to years). The dissolution rate of filaments 17, 117, and 217 may be tailored by controlling the type of biodegradable polymer, the thickness and/or density of the biodegradable polymer, and/or the nature of the biodegradable polymer. In addition, increasing thickness and/or density of a polymeric material will generally slow the dissolution rate of the filaments. Characteristics such as the chemical composition and molecular weight of the biodegradable polymer may also be selected in order to control the dissolution rate of the filaments. In one embodiment, filaments may be made from a biodegradable polymer that is degradable within one year and that has adequate mechanical properties to provide wall apposition and strength for at least six months. Anti-fraying technology may optionally be applied to the ends of filaments to prevent unraveling of the tubular members.

In embodiments, at least a portion of the implantable devices 2, 2′, 2″ may be coated with a therapeutic agent (not shown) such as a controlled-release polymer and/or drug, as known in the art, for reducing the probability of undesired side effects, e.g., restenosis. The therapeutic agent can be of the type that dissolves plaque material forming the stenosis or can be such as an antineoplastic agent, an antiproliferative agent, an antibiotic, an antithrombogenic agent, an anticoagulant, an antiplatelet agent, an anti-inflammatory agent, combinations of the above, and the like. Such drugs can include zotarolimus, rapamyacin, VEGF, TPA, heparin, urokinase, or sirolimus for example. The implantable devices 2, 2′, 2″ may be used for delivering any suitable medications to the walls of a body vessel.

It will be understood that various modifications may be made to the embodiments disclosed herein. In particular, the implantable devices according to the present disclosure may be implanted in any suitable blood vessel. Therefore, the above description should not be construed as limiting, but merely as exemplifications of various embodiments. Those skilled in the art will envision other modifications within the scope and spirit of the claims appended thereto.

1. An implantable device comprising:
a tubular member defining a longitudinal axis and a lumen, the tubular member having:
a plurality of laser cut filaments defining a plurality of openings therebetween;
a distal end portion having a distal diameter;
a proximal end portion having a proximal diameter that is larger than the distal diameter; and
an intermediate portion having an intermediate diameter that is smaller than the distal diameter, wherein when compressed to approximately 30% of a nominal state, a radial force of the tubular member is from about 15 mmHg to about 70 mmHg.

2. The implantable device according to claim 1, wherein the proximal diameter is from about 10 mm to about 14 mm, the distal diameter is from about 4 mm to about 8 mm, and the intermediate diameter is from about 4 mm to about 7 mm.

3. The implantable device according to claim 1, wherein the proximal diameter is larger than the distal diameter by a factor from about 2 to about 3.

4. The implantable device according to claim 1, further comprising:
an attachment member including a plurality of attachment filaments and a hook coupled to the attachment filaments.

5. The implantable device according to claim 4, wherein rotation of the attachment member about the longitudinal axis in a first direction expands the tubular member and rotation in a second direction, opposite the first direction, constrains the tubular member.

6. The implantable device according to claim 4, wherein the tubular member is formed from a non-biodegradable material and the attachment member is formed from a biodegradable material.

7. (canceled)

8. (canceled)

9. (canceled)

10. A method for treating a cerebral dural venous sinus, the method comprising:
collapsing an implantable device into a collapsed configuration, the implantable device including:
a tubular member defining a longitudinal axis and a lumen, the tubular member having:
a plurality of laser cut filaments defining a plurality of openings therebetween;
a distal end portion having a distal diameter;
a proximal end portion having a proximal diameter that is larger than the distal diameter; and
an intermediate portion having an intermediate diameter that is smaller than the distal diameter.
inserting the implantable device into the cerebral dural venous sinus; and
expanding the implantable device inside the cerebral dural venous sinus into an expandable configuration, wherein when compressed to approximately 30% of a nominal state, a radial force of the tubular member is from about 15 mmHg to about 70 mmHg.

11. The method according to claim 10, further comprising:
placing the implantable device within the cerebral dural venous sinus such that the proximal end portion is disposed adjacent a sigmoid sinus of the cerebral dural venous sinus and the distal end portion is disposed adjacent a torcula of the cerebral dural venous sinus.

12. The method according to claim 10, wherein the implantable device further includes:
an attachment member including a plurality of attachment filaments and a hook coupled to the attachment filaments.

13. The method according to claim 12, further comprising:
rotating the attachment member about the longitudinal axis in a first direction to expand the tubular member.

14. The method according to claim 13, further comprising:
rotating the attachment member about the longitudinal axis in a second direction, opposite the first direction to constrain the tubular member.

15. (canceled)

16. (canceled)

17. The method according to claim 10, wherein the proximal diameter is from about 10 mm to about 14 mm, the distal diameter is from about 4 mm to about 8 mm, and the intermediate diameter is from about 4 mm to about 7 mm.

18. (canceled)

19. (canceled)

20. (canceled)

21. The implantable device according to claim 1, wherein when compressed to approximately 30% of the nominal state, the radial force of the tubular member is from about 20 mmHg to about 50 mmHg.

22. The implantable device according to claim 1, wherein in the nominal state, the radial force of the tubular member is from about 10 mmHg to about 30 mmHg.

23. The implantable device according to claim 1, wherein when fully compressed, the radial force of the tubular member is from about 30 mmHg to about 200 mmHg.

24. The implantable device according to claim 23, wherein when fully compressed, the radial force of the tubular member is from about 40 mmHg to about 60 mmHg.

25. The method according to claim 10, wherein when compressed to approximately 30% of the nominal state, the radial force of the tubular member is from about 20 mmHg to about 50 mmHg.

26. The method according to claim 10, wherein in the nominal state, the radial force of the tubular member is from about 10 mmHg to about 30 mmHg.

27. The method according to claim 10, wherein when fully compressed, the radial force of the tubular member is from about 30 mmHg to about 200 mmHg.

28. The method according to claim 27, wherein when fully compressed, the radial force of the tubular member is from about 40 mmHg to about 60 mmHg.
